(19) United States
(12) Patent Application Publication
(10) Pub. No.: US 2007/0095665 A1
(43) Pub. Date: May 3, 2007

(54) Method for enhancing life span and adhesion of electrophoresis deposited electron emission source

(75) Inventors: Chun-Yen Hsiao, Guanyin Township (TW); Shie-Heng Lee, Guanyin Township (TW); Yu-An Li, Guanyin Township (TW); Jin-Lung Tsai, Guanyin Township (TW); Kuei-Wen Cheng, Guanyin Township (TW)

Correspondence Address: HDSL, 4331 STEVENS BATTLE LANE, FAIRFAX, VA, 22033, US

(73) Assignee: Teco Electric & Machinery Co., Ltd.

(21) Appl. No.: 11/266,672

(22) Filed: Nov. 3, 2005

(51) Int. Cl. C07K 1/26; B01D 59/42

(52) U.S. Cl. 204/450

(57) ABSTRACT

A method for enhancing the adhesion and life span of the electrophoresis deposited electron emission source. The method can form a siloxane film on the surface of carbon nanotubes during a one-time electrophoresis deposition process. The siloxane film is then sintered to form a silicon dioxide film on the surface of the electron emission source. This can prevent the intoxication of carbon nanotubes, thereby enhancing the life span of the carbon nanotubes and the adhesion of carbon nanotubes on the electrode layer.

### BACKGROUND OF THE INVENTION

The present invention relates generally to a method for enhancing life span and adhesion of electrophoresis deposited electron emission source, and more particularly to a method that can form a siloxane film on the surface of carbon nanotubes during a one-time electrophoresis deposition process. The siloxane film is then sintered to form a silicon dioxide film on the surface of the electron emission source. This can prevent the intoxication of carbon nanotubes, thereby enhancing the life span of the carbon nanotubes and the adhesion of carbon nanotubes on the electrode layer.

Conventional triode field emission display includes an anode structure and a cathode structure. There is a spacer disposed between the anode structure and the cathode structure, thereby providing a space and a support for the vacuum region between the anode structure and the cathode structure. The anode structure includes an anode substrate, an anode electrode layer, and a phosphorus layer; while the cathode structure includes a cathode substrate, a cathode electrode layer, an electron emission source layer, a dielectric layer, and a gate layer. The gate layer is provided a voltage difference to induce the emission of electrons from the electron emission source layer. The anode electrode layer provides a high voltage to accelerate the electron beam, such that the electron beam can have enough kinetic energy to impinge and excite the phosphorous layer on the anode structure, thereby emitting light. Accordingly, in order to maintain the movement of electrons in the field emission display, a vacuum apparatus is required to keep the vacuum degree of the display being below 10⁻⁵ torr. Therefore, the electrons can have appropriate mean free paths. Meanwhile, one should prevent the electron emission source and the phosphorous layer from polluting and intoxicating the environment. Furthermore, in order for the electrons to accumulate enough energy to impinge the phosphorous powder, a space is required between the two substrates. Consequently, the electrons can be accelerated to impinge the phosphorous layer, thereby exciting the phosphorous layer and emitting light therefrom.

The so-called electron emission source layer is composed of carbon nanotubes. Since carbon nanotubes, discovered by Iijima in 1991 (Nature, 354, 56 (1991)), comprises very good electronic properties, they can be used to build a variety of devices. The carbon nanotubes also has a very large aspect ratio, mostly larger than 500, and a very high rigidity of Young moduli larger than 1000 GPn. In addition, the tips or defects of the carbon nanotubes are of atomic scale. Therefore, the properties of carbon nanotubes described above are considered ideal for building electron field emitter, such as an electron emission source of a cathode structure of a field emission display. Since the carbon nanotubes comprise the physical properties described above, a variety of manufacturing processes can be developed, e.g. screen printing, or thin film processing, so as to pattern the carbon nanotubes for building electronic devices.

However, the art of manufacturing the cathode structure employs carbon nanotubes as an electron emission material, which is fabricated on the cathode electrode layer. The manufacturing process can be a chemical vapor deposition process, or any kind of process that can pattern the photosensitive carbon nanotube solution on any pixel of the cathode electrode layer. Moreover, the cathode structure can also be manufactured by coating the carbon nanotube solution incorporating with a mask. However, there are still limitations on the manufacturing cost and the cubic structure for fabricating the carbon nanotubes on each pixel of the cathode electrode structure, according to the electron emission source structure of the triode field emission display described above. In particular, the homogeneity of large size electron emission source is still very hard to achieve.

Recently, a so-called electrophoresis deposition (EPD) technology has be developed and disclosed, for example, in the United States Patent Publication No. US2003/0102222A 1, entitled “Deposition Method for Nanostructure Materials.” In this patent publication, the carbon nanotubes are formulated into alcoholic suspension solution. On the other hand, ionic salts of magnesium, lanthanum, yttrium, aluminum act as a charger for preparing the electrophoresis solution. The cathode structure to be deposited is then connected with one electrode of the electrophoresis solution. By providing a DC or AC voltage, an electric field is formed in the solution. The chargers in the solution are then dissolved into ions, so as to adhere onto the powder of carbon nanotubes. For this reason, the electric field forms an electrophoresis force to assist the carbon nanotubes depositing onto a certain electrode. In this manner, the carbon nanotubes are disposited onto a patterned electrode. By using the so-called electrophoresis technology described above, the carbon nanotubes are easily deposited onto an electrode and can easily circumvent the limitations of the cathode structure of the triode field emission display. Therefore, this method is widely used in the application of cathode plate fabrication.

Although the electrophoresis deposition method has been widely adopted, part of the mechanism therein still requires improvements. For example, the inventor of the present invention has filed a few patent applications regarding the improved methods for enhancing the adhesion of carbon nanotubes using the electrophoresis deposition technique. In addition, since the electrons generated in the vacuum environment of the vacuum field emission electron emission source are easily reflected or experience second ion bombardment, or are intoxicated by the chemical leaking from the structure, the life span of the vacuum field electron emission source is thus reduced. For this reason, in the United States Patent Application No. US2003/0127960, a protection film is employed and disposed on one end of the carbon nanotubes of the cathode electron emission source, so as to prevent the intoxication of the carbon nanotubes. Accordingly, the inventor of the present invention has developed a carbon nanotubes electrophoresis solution that can form a alkansiloxane compound film on the surface of carbon nanotubes in one electrophoresis deposition process. Under a high temperature sintering process, the siloxane compound film becomes a silicon dioxide film on the electron emission source, thereby preventing the intoxication of carbon nanotubes. In this manner, the life span of the carbon nanotubes is enhanced. Furthermore, the adhesion of carbon nanotubes on the electrode layer is also improved.

### BRIEF SUMMARY OF THE INVENTION

The present invention is to provide a method that can form a protection film on the carbon nanotubes, so as to prevent intoxication of the carbon nanotubes. In this manner, one can enhance the life span and adhesion of the carbon nanotubes on the electron emission source.

In order to achieve the above and other objectives, the method for enhancing life span and adhesion of electrophoresis deposited electron emission source of the present includes the following steps.

First, a semi-manufactured cathode structure is provided. The electrophoresis deposition process is then performed on the semi-manufactured cathode structure. In the electrophoresis deposition process, the cathode structure and the metallic plate are first connected to the electrophoresis electrodes, and are disposed into an electrophoresis solution. A DC pulse voltage is then applied to form an electric field. The carbon nanotubes are then electrophoresis deposited on the cathode structure to form electron emission source. Meanwhile, a siloxane compound film is also formed on the surface of the electron emission source. The electrophoresis solution of the present invention can electrophoresis deposit a electron emission source layer and a siloxane film on the surface of the electron emission source in a one-time deposition process. The electrophoresis solution of the present invention is an alcoholic solution including at least carbon nanotubes power, a charger such as indium salt, and a siloxane compound such as tetraethylorthosilane.

After the deposition process of the cathode structure is completed, the combination is baked with a low temperature so as to remove the residual alcoholic solution on the cathode structure. Meanwhile, the indium chloride charger and the electrolyte hydroxide ions form indium hydroxide, and a siloxane compound film are formed on the surface of the electron emission layer. Next, a sintering process is performed for re-oxidizing the indium hydroxide on the cathode electrode to indium oxide, and for forming silicon dioxide film on the surface of the electron emission source from the siloxane compound film. A protection layer is thus formed on the surface of the carbon nanotubes, which can enhance the adhesion of the carbon nanotubes and the cathode electrode layer.

### BRIEF DESCRIPTION OF THE DRAWINGS

FIG. 1(a) to FIG. 1(g) illustrates the process for manufacturing the semi-manufactured cathode structure.

FIG. 2 illustrates the process for manufacturing the electron emission source on the cathode structure of the present invention.

FIG. 3 illustrates the connection between the cathode structure and a metallic plate.

FIG. 4 illustrates the process for performing the electrophoresis deposition technique, after the cathode structure and the metallic plate are connected.

FIG. 5 illustrates a sectional view of the cathode structure after being electrophoresis deposited with arc discharged carbon nanotubes.

FIG. 6 is a picture of the carbon nanotubes surface taken from a tunneling electron microscopy.

FIG. 7 is another picture of the carbon nanotubes surface taken from a tunneling electron microscopy.

### DETAILED DESCRIPTION OF THE INVENTION

In order to better understanding the features and technical contents of the present invention, the present invention is hereinafter described in detail by incorporating with the accompanying drawings. However, the accompanying drawings are only for the convenience of illustration and description, no limitation is intended thereto.

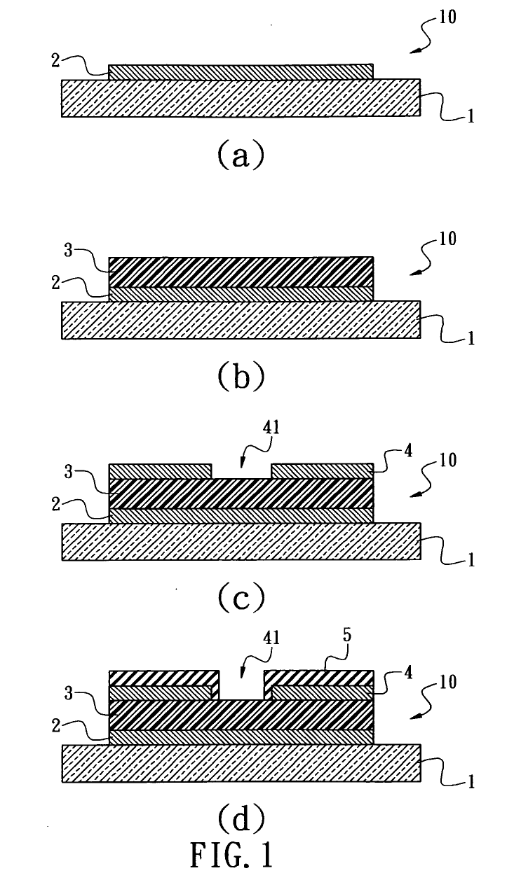

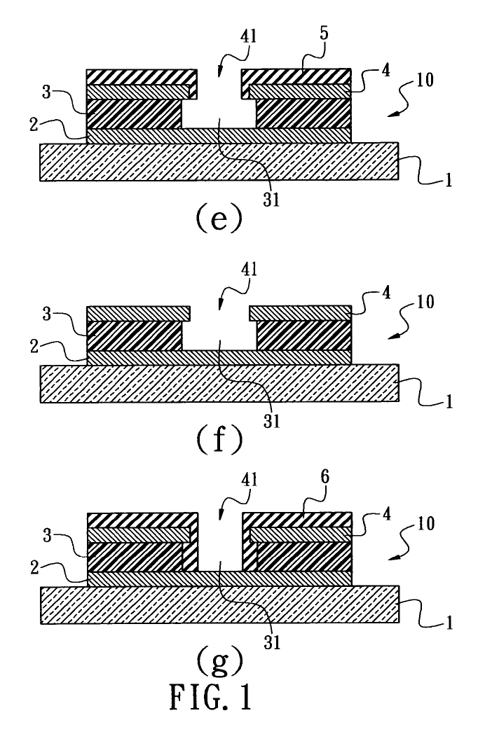

Referring to FIG. 1(a) to FIG. 1(g), a process for manufacturing a semi-manufactured cathode structure, in accordance with the present invention, is illustrated. As shown, the method of the present invention for enhancing the life span and adhesion of electrophoresis deposited carbon nanotubes electron emission source can form a siloxane film on the surface of carbon nanotubes during a one-time electrophoresis deposition process. The siloxane film is then sintered to form a silicon dioxide film on the surface of the electron emission source. This can prevent the intoxication of carbon nanotubes, thereby enhancing the life span of the carbon nanotubes and the adhesion of carbon nanotubes on the electrode layer.

First, a semi-manufactured cathode structure is fabricated. A cathode electrode layer 2 is formed on the surface of a glass substrate 1. A dielectric layer 3 is formed on the surface of the cathode electrode layer 2. Later, a gate electrode layer 4 is formed on the surface of the dielectric layer 3. A sagged region 41 is then formed on the gate layer 4 by lithography technology, thereby exposing the dielectric layer 3. Next, a protection layer 5 is formed on the surface of the gate electrode layer 4. A sagged region 31 is formed on the surface of the dielectric layer 3 to expose the cathode electrode layer 2. The protection layer 5 is then peeled off, and another protection layer 6 is coated covering the dielectric layer 3 and the gate electrode layer. In this manner, the fabrication of the semi-manufactured cathode structure is completed.

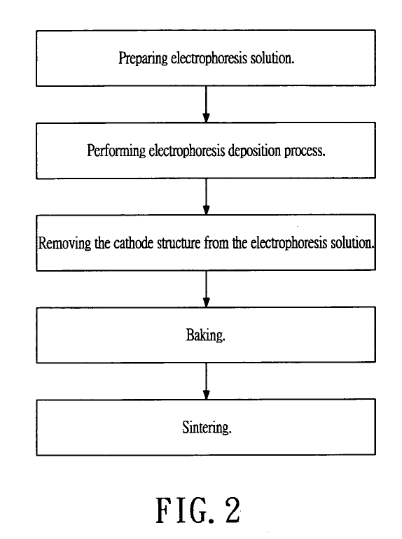

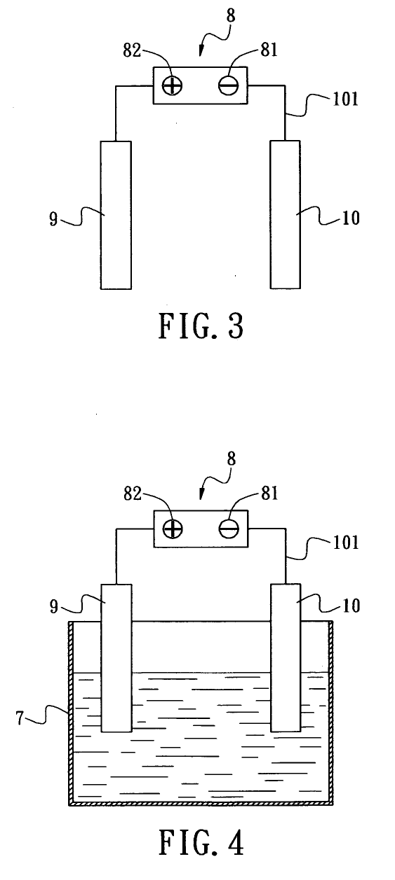

Referring to FIG. 2 to FIG. 4, the process for manufacturing the electron emission source on the cathode structure, and the electrophoresis deposition process before and after the cathode structure and the metallic plate are connected, are illustrated respectively. As shown, after the completion of the semi-manufactured cathode structure mentioned above, the deposition process for manufacturing the carbon nanotube electron emission source of the cathode structure is performed.

First, the electrophoresis solution is prepared by taking alcohol and 1% to 10% (preferably 5%) weight concentration of pure water as a solvent. The water solution is partially provided for ionizing the charger. Then, the carbon nanotubes powder is added. The carbon nanotubes employed in the present invention are multi-wall carbon nanotubes made from arc discharge. The average length and diameter of the carbon nanotubes are below 5 microns and 100 nm, respectively. The weight concentration of the carbon nanotubes is approximately 0.1%˜0.005% (preferably 0.02%). In addition, a charger of weight concentration of approximately 0.1%˜0.005% (preferably 0.01%) is added to the solution. The charger can be any metallic oxide salt that induces conductivity of the electrophoresis, such as indium chloride, indium nitrate, or any other salt comprises tin. In this particular embodiment, the charger is selected to be indium chloride of weight concentration of 0.01% (or between 0.1% to 0.005%). In addition, some acidified siloxane compound is also added. In this particular embodiment, the siloxane compound is the tetraethylorthosilane (TEOS) of weight concentration of 1% to 5% (preferably 3.5%). The so-called acidification process dissolves the liquid polymer TEOS solution into siloxane compound of low molecule weight. The prepared solution is then poured into the electrophoresis tank 7.

After the electrophoresis solution is prepared, one can perform the electrophoresis deposition process. The cathode electrode layer 2 of the field emission cathode structure 10 is connected to the cathode electrode 81 of the electrophoresis electrode 8 via the cathode conducting wire 101. The anode electrode 82 of the electrophoresis electrode 8 is connected to the metallic plate 9. The metallic plate 9 described above can be of a platinum or titanium plate, or a screen plate.

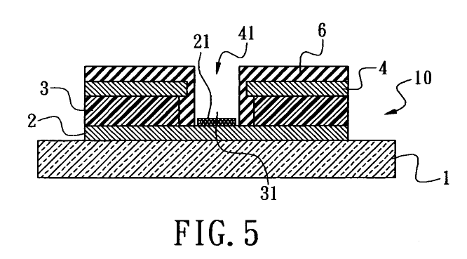

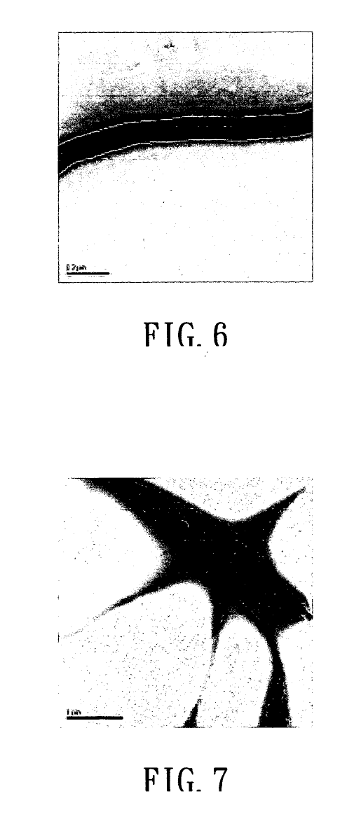

After the cathode structure 10 and the metallic plate 9 are combined, one side of the cathode structure 10 to be electrophoresis deposited is kept parallel to the metallic plate 9 with a fixed distance, and then disposed into the electrophoresis tank 7. A DC pulse voltage from a power supply is provided to the cathode and anode electrodes to form an electric field. The intensity of the electric field can be 0.5˜10 V/cm (preferably 2 V/cm), and the pulse frequency is 300 Hz. The carbon nanotubes are then electrophoresis deposited on the cathode electrode layer 2 to form the electron emission source 21, as shown in FIG. 5. Meanwhile, a tetraethylorthosilane film is formed on the carbon nanotubes of the electron emission source and the surface of the cathode electrode (as shown in FIG. 6 and FIG. 7).

After completing the above deposition process, the cathode structure is moved to an oven and baked under a low temperature of 80° C. to remove the residual alcoholic solution on the cathode structure 10. Now, the indium chloride charger and the electrolyte hydroxyl ions form the indium hydroxide. A sintering process at a temperature of 400° C. is performed to form a tetraethylorthosilane film on the surface of the electron emission source. The indium hydroxide on the cathode electrode layer 2 is further oxidated to form indium oxide. Since indium oxide is conductive, the cathode electrode thus manufactured includes, in addition to the deposited carbon nanotubes, an additional conductive indium oxide layer on the electron emission layer. The tetraethylorthosilane film is further sintered to form a polymer silicon dioxide. The polymer silicon dioxide forms a protection film on the surface of the carbon nanotubes, such that the carbon nanotubes are prevented from intoxication, and the life span of the carbon nanotubes is extended. The polymer silicon dioxide can further enhance the adhesion between the carbon nanotubes and the cathode electrode layer.

Since, any person having ordinary skill in the art may readily find various equivalent alterations or modifications in light of the features as disclosed above, it is appreciated that the scope of the present invention is defined in the following claims. Therefore, all such equivalent alterations or modifications without departing from the subject matter as set forth in the following claims is considered within the spirit and scope of the present invention.

What is claimed is:

1. A method for enhancing adhesion and life span of an electrophoresis deposited electron emission source, comprising:
preparing a semi-manufactured cathode structure;
performing an electrophoresis deposition process to the semi-manufactured cathode structure, wherein the electrophoresis deposition process comprising the steps of connecting the semi-manufactured cathode structure and a metallic plate to electrophoresis electrodes, and disposing the connected semi-manufactured cathode structure and the metallic plate into an electrophoresis solution, wherein the semi-manufactured cathode structure and the metallic plate are parallel and have a fixed distance with each other, and applying a DC (direct current) pulse voltage to form an electric field, thereby manufacturing an electron emission source and forming a siloxane compound film on a surface of the electron emission source;
baking with a low temperature to remove a residual alcoholic solution on the semi-manufactured cathode structure, meanwhile, an indium chloride charger and electrolyte hydroxyl ions form an indium hydroxide, and forming a siloxane compound film on the surface of the electron emission layer, next performing a sintering process for re-oxidizing the indium hydroxide on a cathode electrode to an indium oxide and for forming a silicon dioxide film on the surface of the electron emission source from the siloxane compound film, thereby forming a protection layer on the surface of carbon nanotubes and enhancing the adhesion of the carbon nanotubes and a cathode electrode layer.

2. The method as recited in claim 1, wherein the step for preparing the semi-manufactured cathode structure comprises:
forming the cathode electrode layer on a glass substrate, forming a dielectric layer on the cathode electrode layer, forming a gate layer on the dielectric layer, and forming a sagged region on the gate layer by means of lithography technology to expose the cathode electrode layer;
forming a first protection layer on a surface of the gate layer, etching the dielectric layer to form the sagged region exposing the cathode electrode, and performing peeling operation to the protection layer; and
forming a second protection layer to cover the dielectric layer and the gate layer.

3. The method as recited in claim 1, wherein the cathode electrode layer of the cathode structure connects to a cathode of the electrophoresis electrode via a conducting wire, while an anode of the electrophoresis electrode connects to the metallic plate.

4. The method as recited in claim 1, wherein the metallic plate is a platinum or titanium plate, or a screen plate.

5. The method as recited in claim 1, wherein an intensity of the electric field is about 0.5 to 10 V/m, and a pulse frequency is about 300 Hz.

6. The method as recited in claim 1, wherein an intensity of the electric field is 2 V/m.

7. The method as recited in claim 1, wherein the carbon nanotubes made from arc discharge are multi-wall carbon nanotubes comprising an average length of below 5 microns, and an average diameter below 100 nm.

8. The method as recited in claim 1, wherein a solution contained in a electrophoresis tank comprises alcoholic solvent, pure water, carbon nanotubes powder, charger, and acidified siloxane compound.

9. The method as recited in claim 8, wherein a concentration in weight of the pure water is about 1% to 10%.

10. The method as recited in claim 9, wherein the concentration in weight of the pure water is 5%.

11. The method as recited in claim 8, wherein a concentration in weight of the carbon nanotube powder is about 0.1% to 0.005%.

12. The method as recited in claim 11, wherein the concentration in weight of the carbon nanotube powder is 0.02%.

13. The method as recited in claim 8, wherein the charger is a conductive metallic oxide salt selected from indium chloride, indium nitride, or any other salts comprise tin.

14. The method as recited in claim 13, wherein a concentration in weight of the charger is about 0.1%˜0.005%.

15. The method as recited in claim 14, wherein the concentration in weight of the charger is 0.01%.

16. The method as recited in claim 8, wherein the acidified siloxane compound comprises tetraethylorthosilane.

17. The method as recited in claim 16, wherein a concentration in weight of the tetraethylorthosilane is about 1% to 5%.

18. The method as recited in claim 17, wherein the concentration in weight of the tetraethylorthosilane is 3.5%.

19. The method as recited in claim 1, wherein a baking temperature is about 80° C.

20. The method as recited in claim 1, wherein a sintering temperature is about 400°C.
